(19) United States
(12) Patent Application Publication
(10) Pub. No.: US 2025/0275867 A1
(43) Pub. Date: Sep. 4, 2025

(54) LUBRICATION ASSEMBLY AND METHOD FOR USE THEREOF

(71) Applicant: Alcon Inc., Fribourg (CH)

(72) Inventor: Kevin Canzone, Coto de Caza, CA (US)

(21) Appl. No.: 19/058,606

(22) Filed: Feb. 20, 2025

Related U.S. Application Data
(60) Provisional application No. 63/559,267, filed on Feb. 29, 2024.

(51) Int. Cl. A61F 9/00; A61B 90/20

(52) U.S. Cl. CPC A61F 9/0026; A61B 90/20

(57) ABSTRACT

A system and method for assisting a surgeon performing an ophthalmic procedure on an eye of a patient, the system including a lubrication assembly. The lubrication assembly may include a mounting bracket, a dispenser, a lubricant source, a fluid line, and a first actuator. The dispenser is coupled to the mounting bracket which is configured to be coupled to a surgical microscope. The lubricant source is fluidly coupled to the dispenser by the fluid line for movement of a lubricant between the lubricant source and the dispenser. The first actuator dispenses the lubricant from the dispenser onto the eye of the patient.

### INTRODUCTION

Ophthalmic or ocular surgeries require frequent lubrication of a patient's eye while being performed. The eye of the patient must be constantly lubricated during all types of ophthalmic procedures so as to avoid damage to ocular tissue. Traditionally, while the surgeon is performing the ophthalmic procedure, an assistant or other medical professional is within the vicinity with a dropper or syringe filled with lubricant to manually disperse the lubricant. When the surgeon calls out for more lubricant, the assistant then manually applies it to the patient's eye using the dropper.

### SUMMARY

The present disclosure relates generally to a lubrication assembly for surgical microscopes and a method for use thereof.

In certain embodiments, a lubrication assembly for lubricating an eye of a patient during an ophthalmic procedure is provided. The lubrication assembly includes a mounting bracket configured to be coupled to a surgical microscope, a dispenser coupled to the mounting bracket, a lubricant source fluidly coupled to the dispenser, a fluid line for movement of a lubricant between the lubricant source and the dispenser, and a first actuator for dispensing, from the dispenser, the lubricant onto the eye of the patient.

In certain embodiments, a method for lubricating an eye of a patient during an ophthalmic procedure is provided. The method includes moving a lubrication assembly into a selected position relative to the eye of the patient, dispensing a lubricant from the lubrication assembly onto the eye of the patient, and monitoring a position of the lubrication assembly relative to the eye of the patient for the duration of the ophthalmic procedure.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1 illustrates a surgical setting, according to certain embodiments.

FIG. 2 is a schematic diagram of a lubrication assembly, according to certain embodiments.

FIG. 3A is a magnified cross-sectional side view depicting the lubrication assembly of FIG. 2 coupled to a surgical microscope, according to certain embodiments.

FIG. 3B is a bottom-up view of the surgical microscope and the lubrication assembly of FIG. 3A, according to certain embodiments.

FIG. 4 is a schematic diagram illustrating components of the surgical system, according to certain embodiments.

FIG. 5 is a flow diagram illustrating a technique for lubricating an eye of a patient during an ophthalmic procedure, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

As used herein, the term “surgical setting” may refer to any environment where surgical procedures are performed. For example, the term “surgical setting” may refer to an operating theater or an operating room, with one or more surgeons and operating staff involved in the surgical setting.

As used herein, the term “surgical system” may refer to any surgical system, console, or device for performing a surgical procedure. For example, the term “surgical system” may refer to a surgical tool or system, such as a phacoemulsification console, a laser system, an imaging system, an intraocular lens (IOL) alignment system, a biometer, an optical coherence tomography (OCT) machine, or a vitrectomy console.

As used herein, the term “distal” may refer to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

Although generally described with reference to an ophthalmic surgical setting, the devices and systems described herein may be implemented in other settings and contexts, such as other surgical settings, without departing from the scope of the present application.

Manual application of lubricant to a patient's eye as described above can carry with it several disadvantages. For example, the application of lubricant by the assistant or other medical professional can temporarily block or obstruct the surgeon's view of the patient's eye, leading to inefficient performance of the ophthalmic procedure. Additionally, the assistant applying the lubricant may apply either too much, too little, or may apply lubricant to the wrong portion of the patient's eye, thereby leading to inefficient use of the lubricant, which can be expensive and therefore increase the overall cost of the ophthalmic procedure. Furthermore, because the assistant or surgeon must constantly judge the lubrication state of the patient's eye, this can lead to too much elapsed time between lubrication applications, which can make the ophthalmic procedure more uncomfortable for the patient than is necessary. Alternatively, if too much lubrication is applied, this can cause excessive runoff and obscure the surgeon's view at critical points during a surgical procedure.

Accordingly, the systems described herein may overcome many of the limitations associated with current lubrication techniques.

Certain embodiments described herein provide improved lubrication assemblies and techniques for use thereof during the performance of ophthalmic procedures. More particularly, certain embodiments provide lubrication assemblies that automatically dispense a lubricant or other surgical fluid onto an eye of a patient, which eliminates the need for an assistant to manually dispense lubricant onto the eye and reduces complications associated with insufficient lubricant, or other similar events affecting the eye of the patient during ophthalmic procedures.

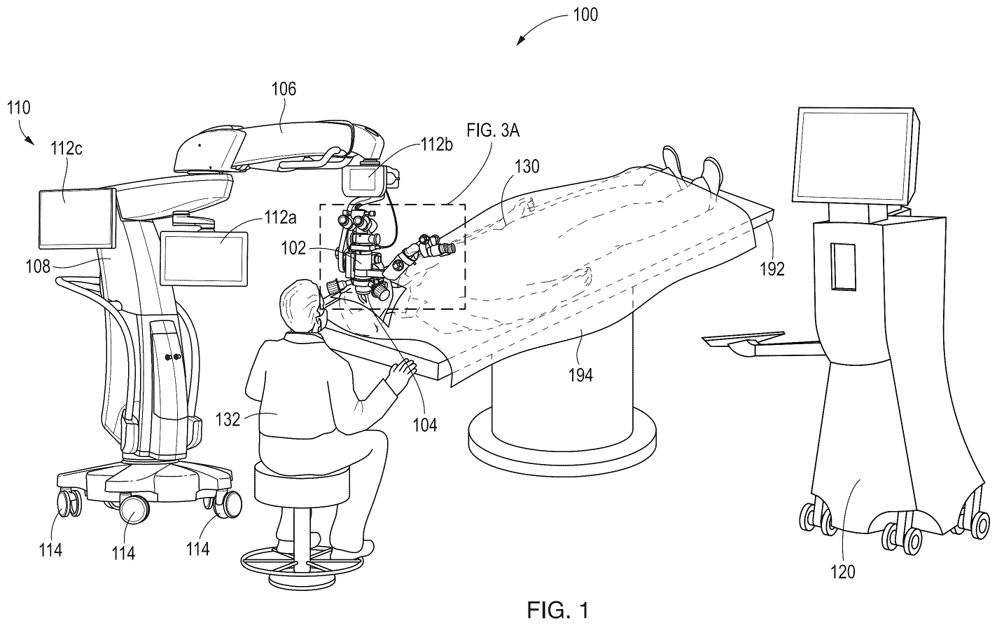

FIG. 1 illustrates an example of a surgical setting 100 in which a surgeon 132 and a patient 130 are present, according to certain embodiments of the present disclosure. The patient 130 is partially covered by a surgical drape 194 and positioned on a patient support table 192, which is illustrated as fixed to the floor of the surgical setting 100 in which the procedure is performed. Although one surgeon 132 is illustrated in FIG. 1, multiple surgeons and/or operating staff may also be present within the surgical setting 100. For example, in certain embodiments, a surgical assistant and/or circulating nurse (i.e., circulator) may also be present within the surgical setting 100.

As shown, the surgeon 132 is operating a surgical microscope system 110 as part of a procedure on the patient 130. Examples of the procedure may include ophthalmic procedures such as, cataract surgeries, vitrectomy surgeries, and/or glaucoma surgeries, but may also include other ophthalmic procedures that involve lubrication or hydration of the eye. The surgical microscope system 110 includes a surgical microscope 102 (e.g., ophthalmic surgical microscope) with a lubrication assembly 104 coupled thereto, an arm 106, a base 108, and video display monitors 112a, 112b, and 112c. The surgical microscope 102 is moveably attached to the arm 106, which is moveably attached to the base 108. As such, the surgeon 132 can manually maneuver the surgical microscope 102 and the lubrication assembly 104 relative to the patient 130 via manipulation of the surgical microscope 102 and/or the arm 106.

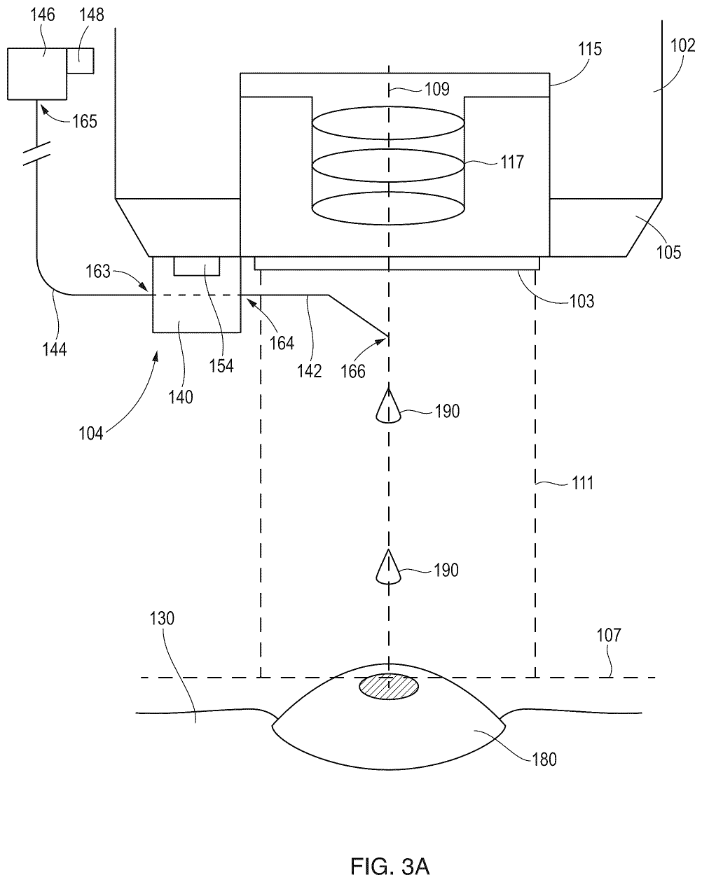

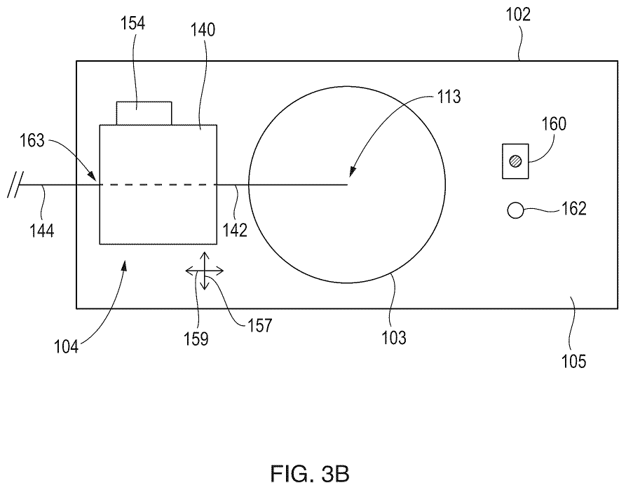

The surgical microscope 102 is operable to hold, move, and position a lubrication assembly 104 in the surgical setting 100 and within a close proximity to an eye 180 of the patient 130 (best seen in FIG. 3A). Although it is currently contemplated that the patient will most frequently be a human patient, in veterinary applications, the “patient” can be a non-human animal, and the term as it appears in this disclosure should be read to encompass either possibility. Additionally, while the surgical microscope 102 is attached to the arm 106 that includes a series of components as shown and described, in certain other embodiments, a robotic arm or other apparatus can be used. For example, rather than a maneuverable arm, a series or plurality of cascaded linear rails may be utilized to secure, move, and provide lubrication assembly 104 to the surgical setting 100 and within close proximity to the eye 180 of the patient 130.

The arm 106 may be supported by any suitable device or system within an operating environment. In the example of FIG. 1, the arm 106 is supported by the base 108. The base 108 can itself be fixed or movable with respect to a structure for supporting the patient 130. For example, the base 108 may be supported by lockable wheels 114 that allow the base 108 to be rolled into position with respect to the patient support table 192. In certain embodiments, however, the arm 106 may be supported by the surgical console 120, the patient support table 192, or another supporting structure within the operating environment. For example, the arm 106 may be movably attached to a supporting mount on a ceiling of the surgical setting 100. Generally, the arm 106 may be coupled to the surgical microscope 102 so that movement of the arm 106 may manipulate both the surgical microscope 102 and the lubrication assembly 104 at the same time.

The configuration of the arm 106 and the base 108 in FIG. 1 is only exemplary, and the exact configuration of the arm 106 and the base 108 can vary considerably in any given embodiment. For example, in certain embodiments, the arm 106 includes elements that provide the arm 106 movement with at least six degrees of freedom. The video display monitors 112 may deliver information and images to medical personnel (e.g., the surgeon 132 and/or the assistant(s)) during the ophthalmic procedure. FIG. 1 illustrates a system with a first display monitor 112a disposed on the arm 106, a second display monitor 112b disposed between the arm 106 and the surgical microscope 102, and a third display monitor 112c disposed on the base 108 for use, for example, by the surgeon 132 and/or the assistant(s). The display monitors 112 can be communicatively coupled with the surgical microscope 102, the surgical console 120, and/or a visualization system within the operating environment. In some embodiments, one or more of the display monitors 112 can receive information (e.g., surgical parameters) and/or images from the surgical console 120, and display the information and images on the one or more display monitors 112. The surgical console 120 can also send signals to the display monitor 112 for performing operations (e.g., starting and stopping video recording).

The surgical microscope system 110 may be in direct or indirect communication with one or more surgical systems, consoles, and/or devices (e.g., integrated in an inter-networked surgical suite) within the surgical setting 100, such as the lubrication assembly 104, the video display monitors 112, and/or the surgical console 120. Examples of suitable surgical systems that may be included in the surgical suite include surgical consoles for performing vitreoretinal procedures, cataract surgeries, corneal transplants, glaucoma surgeries, LASIK surgeries, refractive lens exchanges, trabeculectomies, and refractive surgeries, among other consoles, imaging devices, laser devices, diagnostic devices, and accessories identifiable by those of ordinary skill.

In certain embodiments, the surgical microscope system 110 is a stand-alone device or module that is in wireless or wired communication with the one or more surgical systems physically positioned within the surgical setting 100, such as the surgical console 120. In certain other embodiments, however, the surgical microscope system 110 is integrated within one or more of the surgical systems physically positioned within the surgical setting 100. For example, the surgical microscope system 110 may be integrated with the lubrication assembly 104, the video display monitors 112, and/or the surgical console 120.

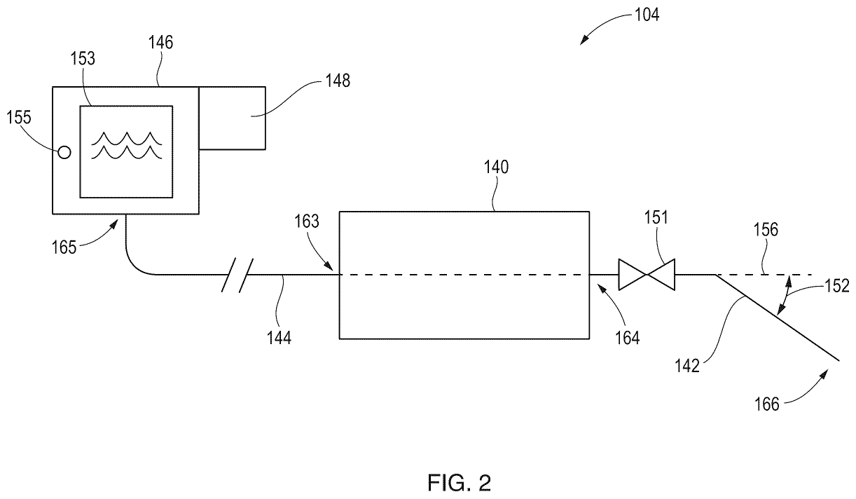

FIG. 2 is a schematic diagram of the lubrication assembly 104 of FIG. 1, according to certain embodiments. The lubrication assembly 104 includes a mounting bracket 140, a dispenser 142, a lubricant source 146, a fluid line 144, and an optional lubricant actuator 148 (also referred to as a “first actuator” herein).

The mounting bracket 140 is configured to couple to the surgical microscope 102 shown in FIG. 1 and attach the dispenser 142 and fluid line 144 thereto. In certain embodiments, the mounting bracket 140 is a dovetail bracket or other suitable rail-type bracket configured to mount to a head 105 of the surgical microscope 102 (best seen in FIG. 3A). The mounting bracket 140 may also be configured to mount to the surgical microscope 102 via one or more screws, an adhesive, or other similar mounting feature. Generally, the mounting bracket 140 is configured to hold the dispenser 142 and the fluid line 144 coupled to the dispenser when the mounting bracket 140 is attached to the surgical microscope 102 such that the dispenser 142 and the fluid line 144 do not obstruct an ophthalmic procedure being performed by the surgeon 132. Generally, a position of the mounting bracket 140 is adjustable, or the position of the dispenser 142 within the mounting bracket 140 is adjustable, such that a position of the dispenser 142 may be manually or automatically adjusted relative to the surgical microscope before or during an ophthalmic procedure.

The dispenser 142 is configured to dispense a lubricant (e.g., a balanced salt solution (BSS)) or other surgical fluid (e.g., a numbing fluid or a dilating fluid) onto the eye 180 of the patient 130. In certain embodiments, the dispenser 142 is a syringe needle. In other embodiments, the dispenser 142 may be an eye dropper, pipette, tubing, nozzle, spout, or other type of fluid conduit configured to dispense fluid in a precise and controlled manner. The dispenser 142 is coupled to the mounting bracket 140 at a connection end 164 of the dispenser 142 and dispenses the lubricant from a dispensing end 166 of the dispenser 142. In certain embodiments, the dispensing end 166 is angled relative to an axis 156 of the dispenser 142 by an angle 152. In certain embodiments, the axis 156 is a major longitudinal axis of the dispenser 142 that extends substantially horizontally relative to a floor of the surgical setting 100 when the dispenser 142 is attached to the surgical microscope 102 via the mounting bracket 140. The angle 152 may be an acute angle (e.g., between 0-90 degrees), a right (e.g., 90 degrees) angle, or an obtuse (e.g., greater than a 90 degrees) angle. The angled orientation of the dispensing end 166 of the dispenser 142 may help facilitate dispensing or dripping of the lubricant onto the eye 180 by preventing the lubricant from collecting at the dispensing end 166, or within the dispenser 142 generally. In other embodiments, however, the dispensing end 166 is disposed along the same longitudinal axis as the connection end 164. Or stated another way, in such embodiments, the angle 152 between the dispensing end 166 and the axis 156 is 0 degrees.

The fluid line 144 is configured to facilitate movement of (or flow of) the lubricant between the lubricant source 146 and the dispenser 142. In other words, the fluid line 144 acts as a conduit for the lubricant. In some embodiments, the fluid line 144 is a cable or other similar tubing provided for supplying the lubricant and/or other fluids, such as water, directly to the dispenser 142 of the lubrication assembly 104. As shown in FIG. 2, a distal end 163 of the fluid line 144 is fluidically coupled to the dispenser 142, while an opposing proximal end 165 of the fluid line 144 is fluidically coupled to the lubricant source 146 or other lubricant reservoir. The fluid line 144 may be at least partially disposed within the mounting bracket 140, and in certain embodiments, may be connected to the connection end 164 of the dispenser 142 within the mounting bracket 140. In certain embodiments, the fluid line 144 is completely disposed exterior to the surgical microscope system 110, or at least partially disposed exterior to the surgical microscope system 110. For example, a portion of the fluid line 144 near the mounting bracket 140 may be disposed exterior to the surgical microscope system 110, and a portion of the fluid line 144 connected to the lubricant source 146 may be disposed within a housing of the surgical microscope 102, the arm 106, and/or the base 108. Generally, the lubricant source 146 may be disposed external to the microscope system 110, and in some examples, attached with one or more components of the microscope system 110, or the lubricant source 146 may be integrated with one or more components of the microscope system 110.

In certain embodiments, the lubrication assembly 104 includes a controlled release valve 151 disposed at the connection end 164 of the dispenser 142, a distal end of the lubricant source 146, and/or at any point of the fluid line 144 or connection elements disposed therebetween to facilitate controllable flow of lubricant from the lubricant source 146 to the dispenser 142, and/or controllable dispensing of the lubricant from the dispenser 142. The controlled release valve 151 is exemplarily shown near the connection end 164 of the dispenser 142 in FIG. 2. The controlled release valve 151 is particularly useful in embodiments of the lubrication assembly 104 without the lubricant actuator 148, where dispensing is facilitated by gravity feed, to control a dispense rate of the gravity feed lubrication assembly 104. Even in embodiments with the lubricant actuator 148, the controlled release valve 151 may be beneficially used to prevent any unwanted dispensing, or leaking, of lubricant from the dispenser 142 when the controlled release valve 151 is disposed at or near the dispenser 142.

In certain embodiments, the controlled release valve 151 is manually controlled. In certain embodiments, the controlled release valve 151 is electronically controlled. In certain embodiments, the controlled release valve 151 may be adjusted (e.g., between an opened and closed position) to allow lubricant to flow from the lubricant source 146 to the dispenser 142, and/or from the dispenser 142 onto the patient's eye 180, at a target flow and/or dispense rate, respectively. The target flow and/or target dispense rate referred to above may be defined by a user. For example, the controlled release valve 151 may be adjusted to allow a target volume (e.g., 1 to 10 milliliters) of the lubricant to be dispensed, in droplets, from the dispenser 142 at a target dispense rate of, for example, once every 1-60 seconds (e.g., once every 1 second, 3 seconds, 5 seconds, 10 seconds, etc.) or once every more than 60 seconds (e.g., once every 90 seconds, 120 seconds, 150 seconds, etc.), or any range associated therewith (e.g., once between very 1 second and 10 seconds, etc.).

In certain embodiments, a level of lubricant within the lubricant source 146 is maintained by manually refilling or replenishing a refillable cartridge 153 or other containment device inserted into the lubricant source 146, or by a direct injection of additional lubricant by the surgeon 132 or the assistant through a port 155 defined on the lubricant source 146 via a separate syringe. In some embodiments, the port 155 may include a self-sealing port, such as a silicone gel or other material. In certain embodiments, the lubricant source 146 is pressure regulated via the lubricant actuator 148 so as to provide a consistent and even flow of the lubricant through the dispenser 142. That is, the lubricant actuator 148 maintains an internal pressure in the lubricant source 146 during dispensing of the lubricant.

The lubricant actuator 148 may be operably coupled to the lubricant source 146 and configured to expel lubricant from the lubricant source 146 to the dispenser 142 via the fluid line 144. That is, the lubricant actuator 148 is configured to act upon the lubricant source 146 so as to force the lubricant from the lubricant source 146 to the dispenser 142, and out of the dispensing end 166 onto the eye 180. In certain embodiments, the lubricant actuator 148 includes a pump, such as a positive displacement pump, for expelling the lubricant from the lubricant source 146 to the dispenser 142. For example, the pump may include a diaphragm pump, a peristaltic pump, a gear pump, a piston pump, or the like. In certain embodiments, the lubricant actuator 148 includes another type of mechanical compression or squeezing device, or other similar actuator. Although the lubricant actuator 148 is shown adjacent to the lubricant source 146 in FIG. 2, the lubricant actuator 148 may be disposed within the lubricant source 146.

In certain embodiments, the lubricant actuator 148 is configured to drive lubricant from the lubricant source 146 to the dispenser 142 such that the lubricant is dispensed from the dispenser 142 at a target dispense rate. For example, a target volume (e.g., 1 to 10 milliliters) of the lubricant may be dispensed, in droplets, from the dispenser 142 at a target dispense rate of, for example, once every 1-60 seconds (e.g., once every 1 second, 3 seconds, 5 seconds, 10 seconds, etc.) or once every more than 60 seconds (e.g., once every 90 seconds, 120 seconds, 150 seconds, etc.), or any range associated therewith (e.g., once between every 1 second and 10 seconds, etc.). In certain embodiments, the lubricant actuator 148 is configured to drive lubricant from the lubricant source 146 to the dispenser 142 at a predetermined, or preset, dispense rate that may be determined automatically or via user input. In certain embodiments, the dispense rate is based on a type of ophthalmic procedure being performed by the surgeon 132. That is, a faster dispense rate may be used for procedures that require more lubrication, and a slower dispense rate may be used for procedures that require less lubrication. In certain embodiments, the dispense rate is based on a size of the eye 180. In further embodiments, the lubricant actuator 148 may be programmed or configured to drive lubricant from the lubricant source 146 to the dispenser 142 such that the lubricant is continuously dispensed from the dispenser 142 in a continuous stream.

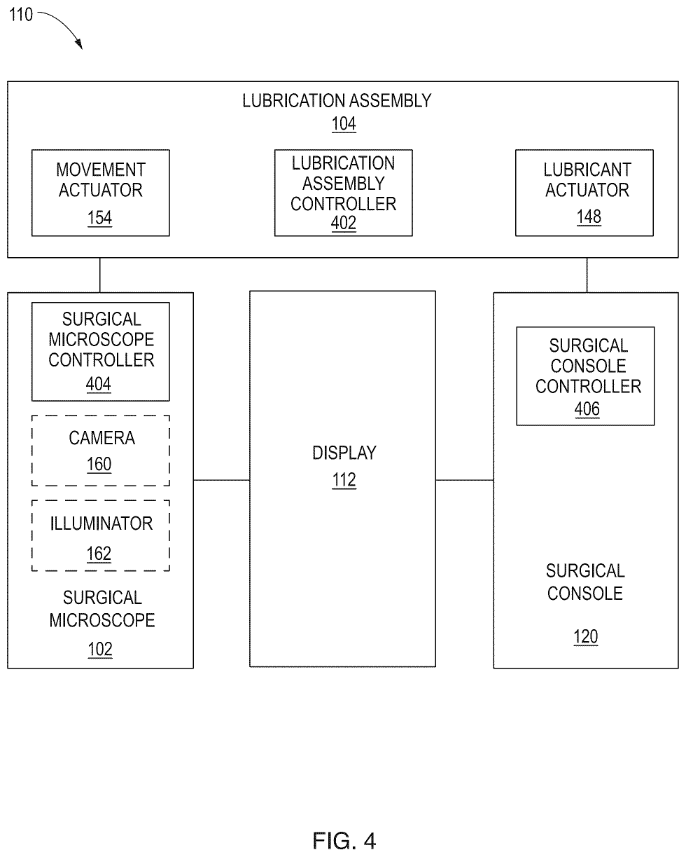

In certain embodiments, the dispense rate when using the lubricant actuator 148 may be set (e.g., preset) by a user (e.g., the surgeon 132 or the assistant) or a corresponding controller, and can be changed during the ophthalmic procedure to adjust the amount of lubrication dispensed onto the eye 180. Dispensing of the lubricant may be manually controlled via various types of user input devices, for example via a manual knob or a valve (e.g., the controlled release valve 151) disposed on the surgical microscope 102 or the surgical console 120 or the lubrication assembly 104, a graphical user interface of the surgical microscope system 110 (e.g., one or more of the display monitor(s) 112) or the surgical console 120, a foot pedal in wired or wireless communication with the lubrication assembly 104 and/or the surgical console 120, or other analog or digital input devices such as a button, switch, keyboard, or the like. In certain embodiments, dispensing of the lubricant may be controlled via audible commands issued by the surgeon 132 or the assistant using known voice command means (e.g., devices and corresponding software) in communication with the lubrication assembly 104. In certain embodiments, the dispense rate is automatically controlled in response to a determined dryness and/or wetness of the eye 180 as described with reference to FIG. 4.

In certain embodiments, dispensing of the lubricant from the lubrication assembly 104 is dictated by a predetermined or time-based schedule. For example, the surgical console 120 or the surgical microscope system 110 may include a clock timer and/or related software that allows the surgeon 132 or the assistant to establish a time interval between each droplet or instance of a predetermined amount of lubricant being dispensed from the lubricant assembly 104. In such embodiments, the lubricant assembly 104 can automatically dispense lubricant according to the established time interval for a predetermined duration of the ophthalmic procedure. If the surgeon 132 or assistant determines that either too much or too little lubricant is being applied, they may adjust the time interval, or the predetermined amount of lubricant being dispensed during each time interval, accordingly until an appropriate amount of lubricant is being applied to the patient's eye 180.

FIG. 3A is a magnified cross-sectional side view depicting the lubrication assembly 104 of FIG. 2 coupled to the surgical microscope 102 of FIG. 1, during use, according to certain embodiments. As shown in FIG. 3A, the surgical microscope 102 and the lubrication assembly 104 are positioned in close proximity to the eye 180 of the patient 130, for example, during an ophthalmic procedure. FIG. 3B is a view from the eye 180 of the patient 130 up towards the surgical microscope 102 and the lubrication assembly 104 of FIG. 3A, according to certain embodiments. FIGS. 3A-3B are described together herein for clarity purposes.

In certain embodiments, the surgical microscope 102 includes a head 105, an optic system 115 with optics 117, a window (or other optical component) 103, a camera 160 (illustrated in FIG. 3B), and an illuminator 162 (illustrated in FIG. 3B). The optic system 115 with optics 117 is disposed within the surgical microscope 102, and the window 103 is centrally disposed at a bottom of the head 105. The camera 160 and the illuminator 162 are disposed radially outward of the window 103 in positions along the head 105 that are unobstructed by the lubrication assembly 104. Although the window 103, the camera 160, and the illuminator 162 are shown in a particular arrangement along the head 105 in the present examples, the window 103, the camera 160, and the illuminator 162 may be positioned in other arrangements in other examples. Further, in certain embodiments, one or both of the camera 160 and the illuminator 162 may be separate devices or components from the surgical microscope 102, and may be attached to the surgical microscope 102 or disposed nearby the head 105 of the surgical microscope 102 in the surgical setting.

The optic system 115 is configured to enable the surgeon 132 using the surgical microscope 102 to observe the eye 180 of the patient 130 at increased magnification and/or under other conditions. For example, the optic system 115 may provide the surgeon 132 with a magnified view of the eye 180 that can be adjusted via manipulation of the optics 117 of optic system 115. In certain embodiments, the optic system 115 is shielded at the bottom of surgical microscope 102 by the window 103, which is configured to prevent debris or other external materials from penetrating and/or affecting the optic system 115 and/or the optics 117. In certain embodiments, the window 103 is transparent and/or coated with an optical coating to enhance transmission.

A field of view (FOV) 111 of the surgical microscope 102, as indicated by the dashed region, may be seen through an eyepiece of the surgical microscope 102 and/or on one or more of the display monitor(s) 112 (FIG. 1). The FOV 111 defines a maximum area or visual field of the optics 117 of the optic system 115. In certain embodiments, a focal plane 107 of the surgical microscope 102, as determined by a focal length of the optics system 115, can be manipulated by the surgeon 132 to move a point of focus along a longitudinal axis 109 of the surgical microscope 102, which may be an optical axis of the optics 117 and/or optic system 115. The focal plane 107 is perpendicular to the longitudinal axis 109 and defines a distance from the window 103 at which an object (e.g., the eye 180) comes into focus.

The illuminator 162 generates and/or emits visible light or other desired wavelengths of light from the head 105 for illuminating the eye 180 or other locations of interest during an ophthalmic procedure. Illumination light reflected from the eye 180 returns to the camera 160, which captures images of the eye 180 and/or dispenser 142 of the lubrication assembly 104. The camera 160 then transmits the images to other components of the surgical microscope system 110 and/or surgical console 120 for processing and, in some embodiments, for display, e.g., as still images or continuous video, on one or more of the display monitor(s) 112, where they can be seen and used by the surgeon 132, an assistant, and/or others involved in the procedure.

In certain embodiments, the images captured by the camera 160 may, for example, be used to continuously track or monitor a position of the eye 180 of the patient 130, the position of the lubrication assembly 104 (i.e., the dispensing end 166) relative to the eye 180 of the patient 130, a lubrication level of the eye 180, and/or to assist in the targeted lubrication of the patient's eye 180, as is further discussed below. In certain embodiments, continuous tracking or monitoring of the position of the eye 180 is carried out via tracking or monitoring of a position of a pupil or vasculature of the eye 180. Although the camera 160 is shown as being disposed through the bottom of surgical microscope 102 in FIG. 3B and configured to directly receive images of the illuminated eye 180, in certain embodiments, the camera is integrated inside surgical microscope 102 or is in optical communication with one or more optics 117 of the optic system 115, and is configured to receive images of the eye 180 via the optics 117.

The lubrication assembly 104 includes the mounting bracket 140, the dispenser 142, the lubricant source 146, the fluid line 144, and in certain embodiments, the lubricant actuator 148, as described with reference to FIG. 2. When the lubrication assembly 104 is mounted to the surgical microscope 102 via the mounting bracket 140, the dispenser 142 and/or the mounting bracket 140 may be adjusted such that the dispensing end 166 extends into the FOV 111, and in certain examples, is positioned along the longitudinal axis 109 between the surgical microscope 102 and the eye 180 (e.g., at or near a center 113 of the window 103). Although the dispenser 142 is disposed in the FOV 111, the dispenser 142 is positioned outside of the working range of focal planes 107 of the surgical microscope 102, such that the dispenser 142 does not obstruct the surgeon 132's view of the eye 180 during the ophthalmic procedure. In other words, when the surgeon 132 is examining the eye 180 through the surgical microscope 102, the dispenser 142 is not visible to the surgeon 132.

When the dispensing end 166 of the dispenser 142 is positioned along longitudinal axis 109, lubricant 190 can be dispensed directly onto an area of the eye 180 on or through which the surgeon 132 is examining the performance of an ophthalmic procedure. Alternatively, when the dispensing end 166 of the dispenser 142 is positioned adjacent to or away from the longitudinal axis 109, lubricant 190 can be dispensed onto the eye 180 at a position adjacent to or away from the area of the eye 180 on or through which the surgeon 132 is examining. When continuous lubrication of eye 180 is desired at the area of the eye 180 on or through which the surgeon 132 is viewing, the lubrication assembly 104 allows the surgeon 132 to continue to examine and/or perform the ophthalmic procedure without visual interruptions for lubrication. For example, the surgeon 132 does not have to stop and wait for the assistant to manually lubricant the eye 180, during which the assistant may also obstruct the surgeon 132's view of the eye 180. The lubrication assembly 104 therefore helps reduce interruptions during ophthalmic procedures and reduces complications associated with insufficient lubrication of the eye 180.

In certain embodiments, the lubrication assembly 104 may further include a movement actuator 154 (which may also be referred to as a “second actuator”) for automatically moving the dispensing end 166 of the dispenser 142 into a selected position relative to the eye 180, as opposed to manual adjustment by a user. The selected position referred to above may be a position that is selected by the user. The movement actuator 154 may include a motor, a drive assembly, or other similar actuator for translating the dispenser 142. In certain embodiments, the movement actuator 154 is configured to linearly move the dispensing end 166 of the dispenser 142 into the selected position by moving the mounting bracket 140 along a y-axis 157 and/or an x-axis 159 (shown in FIG. 3B). As such, the dispensing end 166 may be moved away or toward longitudinal axis 109. In certain embodiments, the movement actuator 154 is coupled to the head 105 of the surgical microscope 102 and the mounting bracket 140 of the lubrication assembly 104. In certain embodiments, the movement actuator 154 is movably coupled to the head 105 and is fixed to the mounting bracket 140, or vice-versa.

Movement of the dispenser 142 via the movement actuator 154 may be controlled via user input by the surgeon 132 or other operating staff (e.g., on the surgical console 120, surgical microscope system 110, or other input device), or automatically by a controller of the lubrication assembly 104, surgical microscope system 110, or surgical console 120 in communication therewith. For example, the surgeon 132 can manually control the movement actuator 154 via a knob on the surgical microscope 102 or the lubrication assembly 104, a graphical user interface of the surgical microscope system 110 (e.g., one or more of the display monitor(s) 112), a foot pedal, or any other analog or digital input device such as a button, switch, keyboard, or the like, or by audible commands issued by the surgeon 132 or the assistant using known voice command means. Alternatively, or in combination therewith, actuation of the movement actuator 154 may be automatically controlled by comparison of the position of the surgical microscope 102, the lubrication assembly 104, and/or the dispensing end 166 with the position of the eye 180, as determined by the surgical microscope system 110 or surgical console 120 in communication therewith.

In certain embodiments, the lubrication assembly 104 may be fixedly attached to the head 105 of the surgical microscope 102. In many cases, though, it may be preferable that the lubrication assembly 104 be conveniently removable and replaceable from the head 105 of the surgical microscope 102. A lubrication assembly 104 that is removable with respect to the surgical microscope allows for the replacement of a malfunctioning lubrication assembly 104, for the removal and sterilization of a lubrication assembly 104 between procedures, for the removal and disposal of a single-use (e.g., disposable) lubrication assembly 104 after a procedure, and/or for other advantages as appropriate depending on the particular application.

FIG. 4 is a schematic system diagram illustrating various example components of the lubrication assembly 104, and the associated surgical microscope system 110 and surgical console 120 of FIG. 1. In the example of FIG. 1, the base 108 provides physical support for the arm 106, which in turn carries the surgical microscope 102 and the lubrication assembly 104, which may be in wired or wireless communication with the surgical console 120. As shown in FIG. 4, the lubrication assembly 104 includes an optional lubrication assembly controller 402, which may be integrated with or in wired or wireless communication with the other components of lubrication assembly 104; the surgical microscope includes an optional surgical microscope controller 404, which may be integrated with or in wired or wireless communication with the other components of surgical microscope 102; and the surgical console 120 includes a surgical console controller 406, which may be integrated with or in wired or wireless communication with the other components of the surgical console 120. The surgical microscope 102 and/or the surgical console 120 are also in wireless or wired communication with the display 112. Note that although certain functions below are described with reference to the surgical console controller 406, such functions may be performed by one or both of the lubrication assembly controller 402 and the surgical microscope controller 404, separately from or in combination with the surgical console controller 406.

Generally, each of the controllers 402, 404, and/or 406 include a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C++ or any other programming language (including assembly languages, hardware description languages, and database programming languages).

The processor may be, or include, a microprocessor, a microcontroller, an embedded microcontroller, a programmable digital signal processor, or any other programmable device operable to receive information from the memory or other devices in communication with the processor and/or controllers 402, 404, 406, and perform one or more operations on the received information. For example, the processor may send instructions to components of the surgical microscope system 110, or other devices or systems in communication with controllers 402, 404, and/or 406, for controlling such devices and systems. The processor may also be operable to output results based on the operations performed thereby. In certain examples, display 112 shows data and other output results provided by the processor of a controller. In some instances, the processor may also be or include an application specific integrated circuit, a programmable gate array, programmable array logic, or any other device of combinations of devices operable to process electric signals.

As shown in FIG. 4, the surgical microscope 102 further includes the illuminator 162, which generates and delivers illumination energy to the eye 180 of the patient 130 to illuminate the eye 180, and the camera 160, which is configured to capture images of the illuminated eye 180. The illumination energy from the illuminator 162 may often be in the form of visible light, but may also include near-infrared or ultraviolet energy, or other illumination energies (including those from laser sources) appropriate to the visualization desired for a given application. The illumination energy projected from the illuminator 162 is reflected from the patient's eye 180 back to the camera 160. This reflected energy may be captured (e.g., converted to digital signals) by one or more sensors of the camera 160. The camera 160 may include, for example, an active-pixel sensor based on a complementary metal-oxide semiconductor (CMOS) sensor.

The digital signals corresponding to images (e.g., image data) captured by the camera 160 may be transmitted to the surgical console controller 406, or other suitable controller, where image analysis software contained therein continuously analyzes the digital signals. In certain embodiments, the image analysis software analyzes the digital signals to determine and/or monitor a lubrication level (e.g., a relative amount of moisture or lubrication) on the surface of the eye 180 of the patient. For example, the image analysis software may determine and/or monitor the general lubrication level of the entire surface of the eye 180, or may determine and/or monitor the lubrication level of one or more particular regions of the eye 180. To determine lubrication levels, the image analysis software can detect and/or monitor amounts of glare within the transmitted image(s), where the amounts of glare correspond to a lubrication level. The glare can be detected and/or monitored for the entire surface of the eye 180, or one or more particular regions of the eye 180. Particular regions of the eye 180 can be identified and monitored by determining a relative position of the eye 180 via tracking of a pupil or vasculature of the eye 180.

In certain embodiments, in response to a determination of a lubrication level, the surgical console controller 406 can send a signal to the lubrication assembly 104 to automatically adjust a dispense rate of lubricant. For example, in certain embodiments, when a detected level (for a particular region or entire surface of the eye 180) of glare drops below a predetermined threshold value, thereby indicating a low level of moisture or lubricant on the surface of the patient's eye 180, the surgical console controller 406 sends a signal to the lubricant actuator 148, or controlled release valve 151, to increase a dispense rate of lubricant. The signal may cause the lubricant actuator 148 to pump lubricant from the lubricant source 146 faster, or may cause the controlled release valve 151 to further open. Conversely, when a large amount of glare has been detected, thereby indicating that the patient's eye 180 has a sufficient amount of lubrication, the surgical console controller 406 sends no signal to the lubricant actuator 148 or controlled release valve 151, or sends a signal to the lubricant actuator 148 or controlled release valve 151 to reduce a dispense rate of lubricant. The signal may cause the lubricant actuator 148 to stop pumping or pump lubricant from the lubricant source 146 slower, or may cause the controlled release valve 151 to further close.

In certain embodiments, the surgical console controller 406 may continuously monitor the amount of glare within the received image data so that lubricant may be added in real-time to the patient's eye 180 during the ophthalmic procedure; however in other embodiments, the surgical console controller 406 periodically detects the amount of glare within the received image data according to a predetermined schedule or time interval so that lubricant is only periodically added during selected moments of the ophthalmic procedure.

In certain embodiments, in response to a determination of a lubrication level, the surgical console controller 406 can also send a signal to the lubrication assembly 104 to automatically adjust a position of the dispensing end 166 of the lubrication assembly 104. For example, in certain embodiments, when a detected level of glare for a particular region of the eye 180 drops below a predetermined threshold value, the surgical console controller 406 sends a signal to the movement actuator 154 to move the lubrication assembly 104 such that the dispenser 142 of the lubrication assembly 104 is disposed over the particular region.

In certain embodiments, instead of automatically adjusting a dispense rate of lubricant from the lubrication assembly 104 or a position of the dispensing end 166 of the lubrication assembly 104 in response to a determination of a lubrication level, the surgical console controller 406 can provide a notification to the surgeon or other operating staff to adjust the dispense rate or reposition the dispensing end 166. For example, in certain embodiments, the surgical console controller 406 may generate and transmit a visual signal to the display 112 to display a graphic or text message for inspection by the surgeon or other operating staff. In certain embodiments, the surgical console controller 406 may generate and transmit an audio signal to a speaker of the surgical console 120, surgical microscope 102, and/or lubrication assembly 104 to emit an audible tone or message for the surgeon or other operating staff.

In certain embodiments, in addition to or separately from monitoring lubrication levels, the surgical console controller 406, or other suitable controller, can monitor or track the position of the dispensing end 166 of the lubrication assembly 104 relative to the patient's eye 180 or portions thereof. For example, the surgical console controller 406 may comprise image analysis software that analyzes digital signals received from the camera 160 to determine and/or monitor a position of the lubrication assembly 104 and thus, a position of the dispensing end 166 of the dispenser 142, relative to the position of the eye 180. Determinations of the position of the dispensing end 166 may be made by comparing a position of, e.g., the dispensing end 166 over the eye 180 and the positions of a pupil or vasculature of the eye 180. In certain embodiments, a relative position of the dispensing end 166 is determined via one or more positional encoders in the lubrication assembly 104.

Monitoring or tracking the position of the dispensing end 166 of the lubrication assembly 104 can be used to maintain a selected position of the lubrication assembly 104 relative to the eye 180, or for modifying an amount of lubricant being applied to a particular region of the eye 180, as described below. In certain embodiments, in response to a determination that the position of the dispensing end 166 of the lubrication assembly 104 over the eye 180 has changed relative to a selected position, the surgical console controller 406 can send a signal to the lubrication assembly 104 to automatically adjust a position of the dispensing end 166. For example, in certain embodiments, when a detected position of the dispensing end 166 is different from a preset or pre-programmed position, the surgical console controller 406 sends a signal to the movement actuator 154 to adjust the detected position to the preset position.

In certain embodiments, the surgical console controller 406 may continuously monitor the position of the dispensing end 166 within the received image data so that the position may be adjusted in real-time to the patient's eye 180 during the ophthalmic procedure; however in other embodiments, the surgical console controller 406 periodically detects the position of the dispensing end 166 within the received image data according to a predetermined schedule or time interval so that the position is only periodically checked/adjusted during selected moments of the ophthalmic procedure.

In certain embodiments, instead of automatically adjusting a position of the dispensing end 166 in response to detecting a change of relative position, the surgical console controller 406 can provide a notification to the surgeon or other operating staff to adjust the position of the lubrication assembly 104. For example, in certain embodiments, the surgical console controller 406 may generate and transmit a visual signal to the display 112 to display a graphic or text message for inspection by the surgeon or other operating staff. In certain embodiments, the surgical console controller 406 may generate and transmit an audio signal to a speaker of the surgical console 120, surgical microscope 102, or lubrication assembly 104 to emit an audible tone or message for the surgeon or other operating staff.

In certain embodiments, the components and/or processing described with reference to FIG. 4 may also be included in, and/or performed by other devices in the surgical settings other than the surgical microscope 102, the surgical microscope system 110, and/or the surgical console 120.

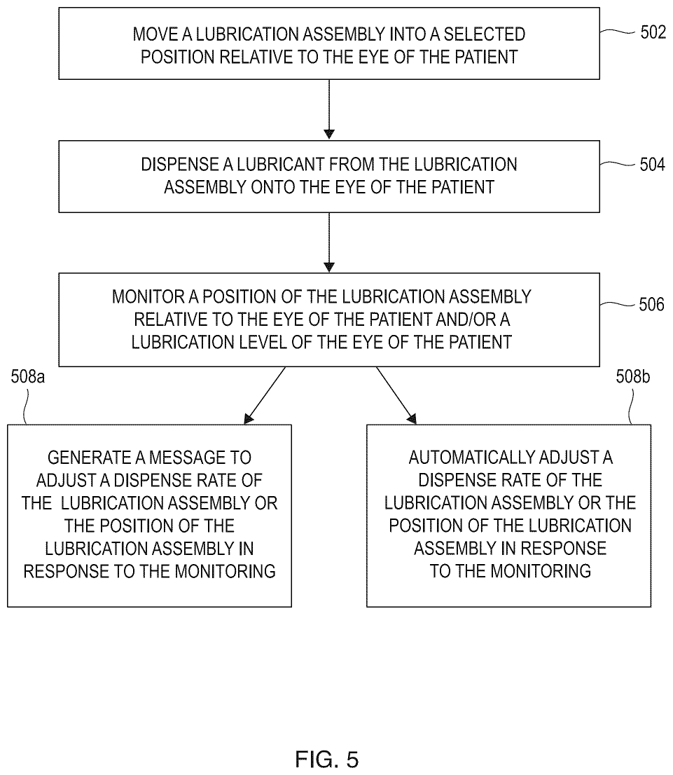

FIG. 5 is a flow diagram illustrating a technique for lubricating an eye of a patient (e.g., the eye 180 of the patient 130) during an ophthalmic procedure, according to certain embodiments.

The technique begins at block 502. At block 502, a lubrication assembly (e.g., lubrication assembly 104) is moved into a selected position relative to the eye 180 of the patient 130. The lubrication assembly 104 may be moved into the selected position by moving the surgical microscope 102 and/or by actuating the movement actuator 154 operatively coupled to the lubrication assembly 104. The lubrication assembly 104 is moved to the selected position over the eye 180 such that the dispensing end 166 is disposed over the eye 180.

At block 504, a lubricant (e.g., lubricant 190) is dispensed from the lubrication assembly 104 onto the eye 180 of the patient 130, as described above with reference to various features in FIGS. 1-4. Generally, the lubricant 190 is dispensed from the dispensing end 166 of the dispenser 142 of the lubrication assembly 104. In certain embodiments, the lubricant 190 may be flowed from the lubricant source 146 to the dispenser 142 via the fluid line 144. For example, the lubricant 190 may be dispensed from the dispenser 142 by activating the lubricant actuator 148 as described with reference to FIG. 2, or by gravity feed. When the lubricant 190 is dispensed using the lubricant actuator 148, a dispense rate of the lubricant 190 is controlled via the lubricant actuator 148 and/or the controlled release valve 151. When the lubricant 190 is dispensed using gravity feed, the dispense rate of the lubricant 190 is controlled via the controlled release valve 151.

At block 506, a position of the lubrication assembly 104 (e.g., dispensing end 166) relative to the eye 180 of the patient 130 and/or a lubrication level of the eye 180 is monitored for a duration of the ophthalmic procedure. The position of the dispensing end 166 relative to the eye 180 and/or the lubrication level of the eye 180 may be monitored using digital signals corresponding to images captured by the camera 160. The digital signals may be communicated to a controller (e.g., the lubrication assembly controller 402, the surgical microscope controller 404, and/or the surgical console controller 406) for further processing and/or analysis.

In certain embodiments, a position of the lubrication assembly 104 (i.e., the dispensing end 166) relative to the eye 180 is determined from the digital signals, in order to determine whether the position of the dispensing end 166 should be adjusted. That is, the position of the dispensing end 166 and/or the eye 180 is monitored to ensure consistent dispensing of the lubricant 190 at a desired area of the eye 180. In certain embodiments, a lubrication or moisture level of the eye is determined from the digital signals, in order to determine whether a dispensing rate or the position of the dispensing end 166 should be adjusted. That is, the lubrication level of the eye 180 is monitored to determine whether to adjust the dispense rate or to move the lubrication assembly 104 to dispense onto a different area of the eye 180 to ensure sufficient lubrication of the eye 180.

At block 508a, in response to the monitoring at block 506, a message (e.g., a notification, a prompt, etc.) is generated and provided to a user (e.g., a surgeon or other operating staff), which may recommend that the user adjust the dispense rate of the lubrication assembly 104 or the position of the lubrication assembly 104. In certain embodiments, the message is provided to the user if the position of the eye 180 changes, the eye 180 or an area thereof needs more lubricant, and/or the lubrication assembly 104 is moved. Examples of a message may include indications of changes in lubrication level (of the eye 180 or area thereof), indications of changes in position, or indications that affirmatively instruct or prompt the surgeon, or operating staff to adjust the dispense rate and/or the position of the dispensing end 166. In certain embodiments, whether the message is provided to the user is determined by at least one of the controllers 402, 404, 406, which process images received via the camera 160.

At block 508b, in addition or alternatively to block 508a, a message (e.g., signal, instruction, etc.) is generated and provided to a corresponding controller in communication with the lubrication assembly 104 (e.g., the lubrication assembly controller 402, the surgical microscope controller 404, and/or the surgical console controller 406) to cause the controller to automatically adjust the dispense rate of the lubrication assembly 104 or the position of the lubrication assembly 104. For example, in certain embodiments, the dispense rate or the position of the dispensing end 166 is automatically adjusted by the lubrication assembly controller 402 if the eye 180 or an area thereof needs more lubricant, the position of the eye 180 changes, and/or the lubrication assembly 104 is moved, based on a message provided to the lubrication assembly controller 402 in response to the monitoring at block 506. In such embodiments, the corresponding controller (e.g., the lubrication assembly controller 402, the surgical microscope controller 404, and/or the surgical console controller 406) sends controlling signals to the lubricant actuator 148 and/or the movement actuator 154 to adjust the dispense rate and/or the position of the dispensing end 166.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A lubrication assembly for lubricating an eye of a patient during an ophthalmic procedure, the lubrication assembly comprising:
a mounting bracket configured to be coupled to a surgical microscope;
a dispenser coupled to the mounting bracket;
a lubricant source fluidly coupled to the dispenser;
a fluid line for movement of a lubricant between the lubricant source and the dispenser; and
a first actuator for dispensing, from the dispenser, the lubricant onto the eye of the patient.

2. The lubrication assembly of claim 1, wherein the lubricant is automatically dispensed from the dispenser at a target dispense rate.

3. The lubrication assembly of claim 2, wherein the target dispense rate is based on a type of the ophthalmic procedure performed.

4. The lubrication assembly of claim 2, wherein the target dispense rate is set by a user.

5. The lubrication assembly of claim 2, wherein the target dispense rate is controlled by a controlled release valve coupled to the dispenser.

6. The lubrication assembly of claim 1, wherein:
the dispenser is disposed in a field of view (FOV) of the surgical microscope; and
the dispenser is outside of a focal plane of the surgical microscope.

7. The lubrication assembly of claim 1 further comprising:
a second actuator for moving the dispenser into a selected position relative to the eye of the patient.

8. The lubrication assembly of claim 1, wherein a dispensing end of the dispenser is positioned at a center of an FOV of the surgical microscope.

9. The lubrication assembly of claim 1, wherein a dispensing end of the dispenser is positioned between the eye of the patient and the surgical microscope.

10. The lubrication assembly of claim 1, wherein the lubricant is automatically dispensed from the dispenser upon actuation of the actuator.

11. A method for lubricating an eye of a patient during an ophthalmic procedure comprising:
moving a lubrication assembly into a selected position relative to the eye of the patient;
dispensing a lubricant from the lubrication assembly onto the eye of the patient;
monitoring a position of the lubrication assembly relative to the eye of the patient and a lubrication level of the eye of the patient for a duration of the ophthalmic procedure; and
generating a message to adjust a dispense rate of the lubrication assembly or the position of the lubrication assembly in response to the monitoring.

12. The method of claim 11, wherein dispensing the lubricant comprises:
automatically dispensing the lubricant from the lubrication assembly at a target dispense rate.

13. The method of claim 11, wherein moving the lubrication assembly comprises:
actuating an actuator coupled to the lubrication assembly to move a dispensing end of the lubrication assembly into the selected position.

14. The method of claim 11, wherein moving the lubrication assembly comprises:
actuating a surgical microscope comprising the lubrication assembly into the selected position.

15. The method of claim 11, wherein monitoring the position of the lubrication assembly comprises:
capturing an image of the patient's eye;
determining a position of the patient's eye from the image;
comparing the position of the lubrication assembly to the position of the patient's eye; and
determining whether to move the lubrication assembly based on the comparison.

16. The method of claim 11, the method further comprising:
providing the message to a user of the lubrication assembly.

17. The method of claim 11, the method further comprising:
providing the message to a controller of the lubrication assembly, wherein providing the message to the controller of the lubrication assembly causes the controller to automatically adjust the dispense rate of the lubrication assembly or the position of the lubrication assembly.

18. A method for lubricating an eye of a patient during an ophthalmic procedure comprising:
moving a lubrication assembly into a selected position relative to the eye of the patient;
dispensing a lubricant from the lubrication assembly onto the eye of the patient;
monitoring a position of the lubrication assembly relative to the eye of the patient and a lubrication level of the eye of the patient for a duration of the ophthalmic procedure; and
automatically adjusting a dispense rate of the lubrication assembly or the position of the lubrication assembly in response to the monitoring.

19. The method of claim 18, wherein monitoring the position of the lubrication assembly relative to the eye of the patient and the lubrication level of the eye of the patient comprises:
determining the eye of the patient needs more of the lubricant.

20. The method of claim 18, wherein monitoring the position of the lubrication assembly relative to the eye of the patient and the lubrication level of the eye of the patient comprises:
determining an area of the eye of the patient needs more of the lubricant.
